(19) United States
(12) Patent Application Publication
(10) Pub. No.: US 2009/0143311 A1
(43) Pub. Date: Jun. 4, 2009

(54) Compositions comprising tripeptides inhibiting ace

(76) Inventors: Luppo Edens, Rotterdam (NL); Cinderella Christina Gerhardt, Vlaardingen (NL); Christianus Jacobus Van Platerink, Vlaardingen (NL); Swen Wolfram, Waldschut-Tiengen (DE)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/919,447

(22) PCT Filed: Mar. 31, 2006
(86) PCT No.: PCT/EP2006/003266

§ 371 Date: Aug. 27, 2008

(30) Foreign Application Priority Data
Apr. 28, 2005 (EP) 05076015.6

(51) Int. Cl. A61K 38/06

(52) U.S. Cl. 514/18

(57) ABSTRACT

The present invention describes the use of MAP and/or ITP or a salt thereof as a nutraceutical, preferably a medicament.

The present invention relates to a novel nutraceutical composition.

The present invention relates to compositions comprising the tripeptides Methionine-Alanine-Proline (Met-Ala-Pro, hereinafter: MAP) and/or Isoleucine-Threonine-Proline (Ile-Thr-Pro, hereinafter: ITP). More specifically, the present invention relates to compositions comprising MAP and/or ITP used for the improvement of health or for the prevention and/or treatment of diseases. The compositions are especially useful for treatment or prevention of high blood pressure (hereinafter: hypertension) and heart failure, or associated conditions such as angina pectoris, myocardial infarction, stroke, peripheral arterial obstructive disease, atherosclerosis, and nephropathy. In another aspect, the present invention relates to the use of MAP and/or ITP in the manufacture of a nutraceutical composition for concomitant consumption in the treatment or prevention of hypertension and heart failure. In still another aspect, the invention relates to a method of treatment or prevention of hypertension and heart failure, or associated such as angina pectoris, myocardial infarction, stroke, peripheral arterial obstructive disease, atherosclerosis, and nephropathy wherein an effective amount of a composition comprising MAP and/or ITP is administered to an individual in need of such treatment.

It is known that hypertension is one of the most important preventable causes of premature death worldwide. Furthermore, even a blood pressure at the top end of the normal range is regarded to increase the risk for premature death. Hypertension is a major risk factor for coronary heart disease and the most important risk factor for stroke. It contributes to approximately half of all cardiovascular disease, which accounted for 16.7 million global deaths in 2002. The risk of cardiovascular disease doubles for every 10 point increase in diastolic blood pressure or every 20 point increase in systolic pressure. In most countries, up to one third of the adults suffer from hypertension. The prevalence of hypertension is increasing with age and this trend is especially prominent in developing countries. Moreover, it is estimated that 40% of hypertensive subjects remain undiagnosed.

Currently, there is no curative therapy available for hypertensive subjects and the main goal of treatment is to lower blood pressure to safer levels. Diet and lifestyle modifications such as more exercise, reduced salt intake, and effective stress management may also represent tools for the prevention of hypertension. This in turn may decrease the requirements for medications, which are commonly associated with side effects ranging from dry cough to loss of energy for activities of daily life. Thus, there is huge demand for prevention and treatment of hypertension by dietary supplements which are safe and not associated with the side effects of drugs currently used for treatment of hypertension.

Currently, ACE inhibitors, angiotensin II receptor antagonists, calcium channel blockers, diuretics, and beta blockers are widely used for treatment of hypertension. ACE inhibitors reduce the levels of angiotensin II, a peptide hormone known to increase blood pressure. Angiotensin II receptor antagonists block binding of angiotensin II to its receptor and thereby exert blood pressure lowering effects. Calcium channel blockers reduce the entry of calcium into cells of the blood vessel wall and thus decrease constriction of blood vessels, which in turn lowers blood pressure. Diuretics lead to Increased urinary excretion of sodium and water, which leads to a reduction of blood pressure. Beta blockers block the action of norepinephrine and epinephrine on beta adrenergic receptors and thereby reduce constriction of blood vessels and lower blood pressure.

The present invention relates to MAP and/or ITP or a salt of MAP and/or a salt of ITP thereof as a nutraceutical, preferably a medicament. The invention also relates to the use of MAP and/or ITP or a salt of MAP and/or a salt of ITP as a nutraceutical preferably a medicament, to the use of MAP and/or ITP or a salt of MAP and/or a salt of ITP for the manufacture of a nutraceutical preferably a medicament, to the use of MAP and/or ITP or a salt of MAP and/or a salt of ITP for the improvement of health or the prevention and/or treatment of diseases, to the use of MAP and/or ITP or a salt of MAP and/or a salt of ITP for the manufacture of a nutraceutical preferably a medicament for the treatment of cardiovascular diseases such as hypertension and heart failure, to the use of MAP and/or ITP or a salt of MAP and/or a salt of ITP for the treatment of pre-diabetes or diabetes, to the use of MAP and/or ITP or a salt of MAP and/or a salt of ITP for the treatment or prevention of obesity, to the use of MAP and/or ITP or a salt of MAP and/or a salt of ITP to increase plasma insulin or to increase the sensitivity for plasma insulin, to the use of MAP and/or ITP or a salt of MAP and/or a salt of ITP to increase plasma insulin or to increase the sensitivity for plasma insulin of type 2 diabetes or pre-diabetes, to the use of MAP and/or ITP or a salt of MAP and/or a salt of ITP to lower post-prandial glucose concentrations in blood of type 2 diabetes or pre-diabetes, to the use of MAP and/or ITP or a salt of MAP and/or a salt of ITP to increase post-prandial insulin secretion in blood of type 2 diabetes or pre-diabetes, to the use of MAP and/or ITP or a salt of MAP and/or a salt of ITP wherein MAP and/or ITP is in the form, of a dietary supplement, to the use of MAP and/or the ITP or a salt of MAP and/or a salt of ITP for the manufacture of a functional food product for the therapeutic treatment of the effects of stress, to the use of MAP and/or ITP or a salt of MAP and/or a salt of ITP in topical application preferably in personal care application and to the use of MAP and/or ITP or a salt of MAP and/or a salt of ITP in feed and pet food. MAP is the preferred tripeptide and is preferred in the uses of the present invention.

Furthermore the present invention relates to a method of treatment of type 1 and 2 diabetes, and for the prevention of type 2 diabetes in those individuals with pre-diabetes, or impaired glucose tolerance (IGT) which comprises administering to a subject in need of such treatment MAP and/or ITP or a salt of MAP and/or a salt of ITP and to a method of treatment of people that suffer of hypertension or heart failure or the prevention thereof which comprises administering to a subject in need of such treatment MAP and/or ITP or a salt of MAP and/or a salt of ITP.

According to a further aspect of the invention a method of chemical synthesis of MAP and/or ITP or a salt of MAP and/or a salt of ITP is disclosed. Moreover the present invention relates to a medicament comprising MAP and/or ITP or a salt of MAP and/or a salt of ITP as active ingredient, a dietary supplement comprising MAP and/or ITP or a salt of MAP and/or a salt of ITP as active ingredient, a food comprising MAP and or ITP or a salt of MAP and/or a salt of ITP as active ingredient, a composition comprising MAP and/or ITP or a salt of MAP and/or a salt of ITP as medicament or for health benefits, a composition wherein the health benefit is the treatment of the effects of stress, preferably the composition is a food or feed, a composition comprising MAP and/or ITP or a salt of MAP and/or a salt of ITP for the use as a topical agent preferably for use in personal care and to a composition which is a lotion, a gel or an emulsion.

In accordance with the present invention it has surprisingly been found that both MAP and ITP inhibit angiotensin I converting enzyme (ACE) and thus, exhibit blood pressure lowering effects. Inhibition of ACE results in reduced vasoconstriction, enhanced vasodilation, improved sodium and water excretion, which in turn leads to reduced peripheral vascular resistance and blood pressure and improved local blood flow. Thus, the present compositions are particularly efficacious for the prevention and treatment of diseases that can be influenced by ACE inhibition, which include but are not limited to hypertension, heart failure, angina pectoris, myocardial infarction, stroke, peripheral arterial obstructive disease, atherosclerosis, nephropathy, renal insufficiency, erectile dysfunction, endothelial dysfunction, left-ventricular hypertrophy, diabetic vasculopathy, fluid retention, and hyperaldosteronism. The compositions may also be useful in the prevention and treatment of gastrointestinal disorders (diarrhea, irritable bowel syndrome), inflammation, diabetes mellitus, obesity, dementia, epilepsy, geriatric confusion, and Meniere's disease. Furthermore, the compositions may enhance cognitive function and memory (including Alzheimer's disease), satiety feeling, limit ischemic damage, and prevent reocclusion of an artery after by-pass surgery or angioplasty. Diabetes mellitus is a widespread chronic disease that hitherto has no cure. The incidence and prevalence of diabetes mellitus is increasing exponentially and it is among the most common metabolic disorders in developed and developing countries. Diabetes mellitus is a complex disease derived from multiple causative factors and characterized by impaired carbohydrate, protein and fat metabolism associated with a deficiency in insulin secretion and/or insulin resistance. This results in elevated fasting and postprandial serum glucose concentrations that lead to complications if left untreated. There are two major categories of the disease, insulin-dependent diabetes mellitus (IDDM, T1DM) and non-insulin-dependent diabetes mellitus (NIDDM, T2DM). T1DM=type 1 diabetes mellitus. T2DM=type 2 diabetes mellitus.

T1DM and T2DM diabetes are associated with hyperglycemia, hypercholesterolemia and hyperlipidemia. The absolute insulin deficiency and insensitivity to insulin in T1DM and T2DM, respectively, leads to a decrease in glucose utilization by the liver, muscle and the adipose tissue and to an increase in the blood glucose levels. Uncontrolled hyperglycemia is associated with increased and premature mortality due to an increased risk for microvascular and macrovascular diseases, including nephropathy, neuropathy, retinopathy, hypertension, stroke, and heart disease. Recent evidence showed that tight glycemic control is a major factor in the prevention of these complications in both T1DM and T2DM. Therefore, optimal glycemic control by drugs or therapeutic regimens is an important approach for the treatment of diabetes.

Therapy of T2DM initially involves dietary and lifestyle changes, when these measures fail to maintain adequate glycemic control the patients are treated with oral hypoglycemic agents and/or exogenous insulin. The current oral pharmacological agents for the treatment of T2DM include those that potentate insulin secretion (sulphonylurea agents), those that improve the action of insulin in the liver (biguanide agents), insulin-sensitizing agents (thiazolidinediones) and agents which act to inhibit the uptake of glucose (α-glucosidase inhibitors).

However, currently available agents generally fail to maintain adequate glycemic control in the long term due to progressive deterioration of hyperglycemia, resulting from progressive loss of pancreatic cell function. The proportion of patients able to maintain target glycemia levels decreases markedly over time necessitating the administration of additional/alternative pharmacological agents. Furthermore, the drugs may have unwanted side effects and are associated with high primary and secondary failure rates. Finally, the use of hypoglycemic drugs may be effective in controlling blood glucose levels, but may not prevent all the complications of diabetes. Thus, current methods of treatment for all types of diabetes mellitus fail to achieve the ideals of normoglycemia and the prevention of diabetic complications.

Therefore, although the therapies of choice in the treatment of T1DM and T2DM are based essentially on the administration of insulin and of oral hypoglycemic drugs, there is a need for a safe and effective nutritional supplement with minimal side effects for the treatment and prevention of diabetes. Many patients are interested in alternative therapies which could minimize the side effects associated with high-dose of drugs and yield additive clinical benefits. Patients with diabetes mellitus have a special interest in treatment considered as “natural” with mild anti-diabetic effects and without major side effects, which can be used as adjuvant treatment. T2DM is a progressive and chronic disease, which usually is not recognized until significant damage has occurred to the pancreatic cells responsible for producing insulin (β-cells of islets of Langerhans). Therefore, there is an increasing interest in the development of a dietary supplement that may be used to prevent β-cell damage and thus, the progression to overt T2DM in people at risk especially in elderly who are at high risk for developing T2DM. Protection of pancreatic P-cells may be achieved by decreasing blood glucose and/or lipid levels as glucose and lipids exert damaging effects on β-cells. The reduction of blood glucose levels can be achieved via different mechanisms, for example by enhancing insulin sensitivity and/or by reducing hepatic glucose production. The reduction of blood lipid levels can also be achieved via different mechanisms, for example by enhancing lipid oxidation and/or lipid storage. Another possible strategy to protect pancreatic β-cells would be to decrease oxidative stress. Oxidative stress also causes β-cell damage with subsequent loss of insulin secretion and progression to overt T2DM.

Therefore, T2DM is a complicated disease resulting from coexisting defects at multiple organ sites: resistance to insulin action in muscle and adipose tissues, defective pancreatic insulin secretion, unrestrained hepatic glucose production. Those defects are often associated with lipid abnormalities and endothelial dysfunction. Given the multiple pathophysiological lesions in T2DM, combination therapy is an attractive approach to its management.

The present invention relates to novel nutraceutical compositions comprising MAP and/or ITP. The nutraceutical compositions comprising MAP and/or ITP can also comprise hydrolysate, unhydrolysed proteins and carbohydrates as the active ingredients for the treatment or prevention of diabetes mellitus, or other conditions associated with impaired glucose tolerance such as syndrome X. In another aspect the present invention relates to the use of such compositions as a nutritional supplement for the said treatment or prevention, e.g., as an additive to a multi-vitamin preparations comprising vitamins and minerals which are essential for the maintenance of normal metabolic function but are not synthesized in the body. In still another aspect, the invention relates to a method for the treatment of both type 1 and 2 diabetes mellitus and for the prevention of T2DM in those individuals with pre-diabetes, or impaired glucose tolerance (IGT) or obesity which comprises administering to a subject in need of such treatment MAP and/or ITP and protein hydrolysates or unhydrolysed proteins and/or carbohydrates.

The compositions of the present invention are particularly intended for the treatment of both T1DM and T2DM, and for the prevention of T2DM in those individuals with pre-diabetes, or impaired glucose tolerance (IGT).

The present invention relates to a composition which comprises MAP and/or ITP and optionally a protein hydrolysate.

Furthermore this composition comprises an amino acid, preferably the amino acid is leucine. The MAP and/or ITP, and optionally protein hydrolysate is advantageously used to increase plasma insulin in blood, preferably for type 2 diabetes or pre-diabetes.

Surprisingly it is found that this MAP and/or ITP can be used for type 2 diabetes or prediabetes, preferably to lower post-prandial glucose concentrations or to increase post-prandial insulin secretion in blood.

The compositions comprising a combination of MAP and/or ITP and protein hydrolysates or unhydrolysed proteins and/or carbohydrates synergistically stimulate insulin secretion and increase glucose disposal to insulin sensitive target tissues such as adipose tissue, skeletal muscle and liver and, thus, provide synergistic effects in the treatment of diabetes mellitus.

It is generally recognised that stress-related diseases, and the negative effects of stress upon the body, have a significant impact upon many people. In recent years the effects of stress, and its contribution towards various the development of various diseases and conditions, has gained wider acceptance in the medical and scientific community. Consumers are now becoming increasingly aware of these potential problems and are becoming increasingly interested in reducing or preventing the possible negative impact of stress on their health.

It is a further object of the invention to provide a food product, or an ingredient which can be incorporated therein, which is suitable for use in helping the body deal with the effects of stress.

It is a further object to provide a food product having a high concentration of an ingredient which provides a health benefit, such as helping the body deal with the negative effects of stress.

According to an aspect the present invention provides the use of the tripeptide MAP and/or the tripeptide ITP and/or salts thereof for the manufacture of a functional food product for the therapeutic treatment of the effects of stress.

Certain peptides are known to exhibit anti-stress effects. The tripeptides MAP and ITP and/or the salts thereof are therefore believed to be very suitable for use in providing such a health benefit. The person skilled in the art is well aware of how to determine such properties for a material.

The term nutraceutical as used herein denotes the usefulness in both the nutritional and pharmaceutical field of application. Thus, the novel nutraceutical compositions can find use as supplement to food and beverages, and as pharmaceutical formulations or medicaments for enteral or parenteral application which may be solid formulations such as capsules or tablets, or liquid formulations, such as solutions or suspensions. As will be evident from the foregoing, the term nutraceutical composition also comprises food and beverages containing MAP and/or ITP and optionally protein hydrolysates or unhydrolysed proteins and/or carbohydrates as well as supplement compositions, for example dietary supplements, containing the aforesaid active ingredients.

The term dietary supplement as used herein denotes a product taken by mouth that contains a “dietary ingredient” intended to supplement the diet. The “dietary ingredients” in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders. They can also be in other forms, such as a bar, but if they are, information the label of the dietary supplement will in general not represent the product as a conventional food or a sole item of a meal or diet.

MAP and/or ITP may be made by hydrolysis or fermentation of any suitable substrate containing the fragments MAP and/or ITP. Advantageously the protein substrate contains both fragments MAP and/or ITP. Preferably the protein substrate is casein or milk. The tripeptides MAP (Met-Ala-Pro) and ITP (Ile-Thr-Pro) can also be made by chemical synthesis using conventional techniques.

In accordance with the present invention it has surprisingly been found that a composition comprising MAP and/or ITP stimulate pancreatic insulin secretion and enhance glucose disposal to insulin sensitive target tissues. Therefore, compositions comprising MAP and/or ITP can be used to prevent or treat both T1DM and T2DM, and for the prevention of T2DM in those individuals with pre-diabetes, impaired glucose tolerance (IGT).

The use of combinations of MAP and/or ITP and protein hydrolysates or unhydrolysed proteins and/or carbohydrates, which individually exert different mechanisms of action are effective in achieving and maintaining target blood glucose levels in diabetic patients.

The combinations of the active ingredients identified above have been conceived because of their different actions, to take advantage of synergistic and multiorgan effects. Owing to distinct mechanisms of action of the individual active ingredients the combinations not only improve glycemic control, but also result in lower drug dosing in some settings and minimize adverse effects. Because of their distinct mechanisms and sites of action, the specific combinations of dietary supplements discussed above also take advantage of synergistic effects to achieve a degree of glucose lowering greater than single agents can accomplish. Thus, although the therapies of choice in the therapeutic treatment of T1DM and T2DM is based essentially on the administration of insulin and of oral hypoglycemic drugs, appropriate nutritional therapy is also of major importance for the successful treatment of diabetics. A multi-vitamin and mineral supplement may be added to the nutraceutical compositions of the present invention to obtain an adequate amount of an essential nutrient missing in some diets. The multi-vitamin and mineral supplement may also be useful for disease prevention and protection against nutritional losses and deficiencies due to lifestyle patterns and common inadequate dietary patterns sometimes observed in diabetes. Moreover, oxidant stress has been implicated in the development of insulin resistance. Reactive oxygen species may impair insulin stimulated glucose uptake by disturbing the insulin receptor signaling cascade. The control of oxidant stress with antioxidants such as α-tocopherol (vitamin E) ascorbic acid (vitamin C) may be of value in the treatment of diabetes. Therefore, the intake of a multi-vitamin supplement may be added to the above mentioned active substances to maintain a well balanced nutrition.

Furthermore, the combination of MAP and/or ITP with minerals such as magnesium (Mg²⁺), Calcium (Ca²⁺) and/or potassium (K⁺) may be used for the improvement of health and the prevention and/or treatment of diseases including but not limited to cardiovascular diseases and diabetes.

In a preferred aspect of the invention, the nutraceutical composition of the present invention contains MAP and/or ITP and protein hydrolysates. MAP and/or ITP suitably is present in the composition according to the invention in an amount to provide a daily dosage from about 0.001 g per kg body weight to about 1 g per kg body weight of the subject to which it is to be administered. A food or beverage suitably contains about 0.05 g per serving to about 50 g per serving of MAP and/or ITP. If the nutraceutical composition is a pharmaceutical formulation such formulation may contain MAP and/or ITP in an amount from about 0.001 g to about 1 g per dosage unit, e.g., per capsule or tablet, or from about 0.035 g per daily dose to about 70 g per daily dose of a liquid formulation. Protein hydrolysates suitably are present in the composition according to the invention in an amount to provide a daily dosage from about 0.01 g per kg body weight to about 3 g per kg body weight of the subject to which it is to be administered. A food or beverage suitably contains about 0.1 g per serving to about 100 g per serving of protein hydrolysates. If the nutraceutical composition is a pharmaceutical formulation such formulation may contain protein hydrolysates in an amount from about 0.01 g to about 5 g per dosage unit, e.g., per capsule or tablet, or from about 0.7 g per daily dose to about 210 g per daily dose of a liquid formulation.

In another preferred aspect of the intervention the composition contains MAP and/or ITP as specified above and unhydrolysed proteins. Unhydrolysed proteins suitably are present in the composition according to the invention in an amount to provide a daily dosage from about 0.01 g per kg body weight to about 3 g per kg body weight of the subject to which it is to be administered. A food or beverage suitably contains about 0.1 g per serving to about 100 g per serving of unhydrolysed proteins. If the nutraceutical composition is a pharmaceutical formulation such formulation may contain unhydrolysed proteins in an amount from about 0.01 g to about 5 g per dosage unit, e.g., per capsule or tablet, or from about 0.7 g per daily dose to about 210 g per daily dose of a liquid formulation.

In yet another preferred aspect of the intervention the composition contains MAP and/or ITP and protein hydrolysates or unhydrolysed proteins as specified above and carbohydrates. Carbohydrates suitably are present in the composition according to the invention in an amount to provide a daily dosage from about 0.01 g per kg body weight to about 7 g per kg body weight of the subject to which it is to be administered. A food or beverage suitably contains about 0.5 g per serving to about 200 g per serving of carbohydrates. If the nutraceutical composition is a pharmaceutical formulation such formulation may contain carbohydrates in an amount from about 0.05 g to about 10 g per dosage unit, e.g., per capsule or tablet, or from about 0.7 g per daily dose to about 490 g per daily dose of a liquid formulation.

Preferred nutraceutical compositions of the present invention comprise MAP and/or ITP and protein hydrolysates or unhydrolysed proteins and/or carbohydrates, especially the combinations of

MAP and/or ITP and protein hydrolysates;
MAP and/or ITP and protein hydrolysates and carbohydrates;
MAP and/or ITP and unhydrolysed proteins;
MAP and/or ITP and unhydrolysed proteins and carbohydrates;

Most preferred is the combination of MAP and/or ITP and protein hydrolysates.

### Dosage Ranges (for a 70 kg Person)

MAP and/or ITP: 0.005-70 g/day
Protein hydrolysates: 0.07-210 g/day
Unhydrolysed proteins: 0.07-210 g/day
Carbohydrates: 0.1-490 g/day

The tripeptides MAP (Met-Ala-Pro) and ITP (Ile-Thr-Pro) can be made by a variety of methods including chemical synthesis, enzymatic hydrolysis and fermentation of protein containing solutions.

The identification of biologically active peptides in complex mixtures such as protein hydrolysates or liquids resulting from fermentation is a challenging task. Apart from the basic questions: are we using the right protein substrate, are we using the right enzyme, are we using the right microbial culture, several biologically active peptides can be expected to be present in complex samples containing thousands of peptides. The traditional identification approaches employing repeated cycles of high-performance liquid chromatographic (HPLC) fractionation and biochemical evaluation are generally time consuming and prone to losses of the biologically active peptides present making the detection of relevant bio-activity extremely difficult. In the present work very sophisticated equipment was used and many different protein hydrolysates and fermentation broths were screened finally leading us to the identification of the two novel peptides MAP and ITP which have ACE inhibitory properties. In our approach a continuous flow biochemical assay was coupled on-line to an HPLC fractionation system. The HPLC column effluent was split between a continuous flow ACE bioassay and a chemical analysis technique (mass spectrometry). Crude hydrolysates and fermentation broths were separated by HPLC, after which the presence of biologically active compounds was detected by means of the on-line biochemical assay. Mass spectra were recorded continuously so that structural information was immediately available when a peptide shows a positive signal on the biochemical assay. The tripeptides MAP and ITP as identified by the above mentioned approach can be produced by various methods including economically viable production routes. Production via chemical synthesis is possible using conventional techniques as for instance described in “Peptides: Chemistry and Biology” by N. Sewald and H. D. Jakubke, Eds. Wiley-VCH Verlag GmbH, 2002, Chapter 4. Particular cost-effective methods of chemical peptide synthesis suitable for large-scale production are based on the use of alkylchloroformates or pivaloyl chloride for the activation of the carboxylic group combined with the use of methyl esters for C-terminal protection and benzyloxycarbonyl (Z) or tert-butyloxycarbonyl groups for N-protection. For instance, in the case of MAP, L-proline methylester can be coupled with isobutylchloroformate-activated Z-Ala; the resulting dipeptide can be Z-deprotected through hydrogenolysis using hydrogen and Pd on C and coupled again with isobutylchloroformate-activated Z-Met; of the resulting tripeptide the methyl ester is hydrolyzed using NaOH and after Z-deprotection by hydrogenolysis the tripeptide Met-Ala-Pro is obtained. Similarly, Ile-Thr-Pro can be synthesized but during the coupling reactions the hydroxy function of Thr requires benzyl-protection; in the final step this group is then simultaneously removed during the Z-deprotection.

MAP and/or ITP may also be made by enzymatic hydrolysis or by fermentative approaches using any protein substrate containing the amino acid sequences MAP and/or ITP. Advantageously the protein substrate contains both fragments MAP and ITP. Preferred protein substrates for such enzymatic or fermentative approaches are bovine milk or the casein fraction of bovine milk. Through optimisation of the fermentation or hydrolysis conditions, the production of the biologically active molecules MAP and/or ITP may be maximised. The skilled person trying to maximise the production will know how to adjust the process parameters, such as hydrolysis/fermentation time, hydrolysis/fermentation temperature, enzyme/microorganism type and concentration etc.

MAP and/or ITP or compositions comprising MAP and/or ITP are advantageously hydrolysates and preferably made according to a process involving the following steps:
(a) enzymatic hydrolysis of a suitable protein substrate comprising MAP or ITP in its amino acid sequence resulting in a hydrolysed protein product comprising the tripeptides MAP and/or ITP;
(b) separation from the hydrolysed protein product of a fraction rich in tripeptide MAP and/or the tripeptide ITP; and optionally
(c) concentrating and/or drying the fraction from step b) to obtain a concentrated liquid or a solid rich in tripeptide MAP and/or the tripeptide ITP.

The enzymatic hydrolysis step (a) may be any enzymatic treatment of the suitable protein substrate leading to hydrolysis of the protein resulting in liberation of MAP and/or ITP tripeptides. Although several enzyme combinations can be used to release the desired tripeptides from the protein substrate, the preferred enzyme used in the present process is a proline specific endoprotease or a proline specific oligopeptidase. A suitable protein substrate may be any substrate encompassing the amino acid sequence MAP and/or ITP. Protein substrates known to encompass MAP are, for example, casein, wheat gluten, sunflower protein isolate, rice protein, egg protein. Suitable protein substrates preferably encompass the amino acid sequences AMAP or PMAP as occur in beta-casein bovine, the alpha-gliadin fraction of wheat gluten and in the 2S fraction of sunflower protein isolate.

The casein substrate may be any material that contains a substantial amount of beta-casein and alpha-s2-casein. Examples of suitable substrates are milk as well as casein, casein powder, casein powder concentrates, casein powder isolates, or beta-casein, or alpha-s2-casein. Preferably a substrate that has a high content of casein, such as casein protein isolate (CPI).

The enzyme may be any enzyme or enzyme combination that is able to hydrolyse protein such as beta-casein and/or alpha-s2-casein resulting in the liberation of one or more of the tripeptides of MAP and/or ITP.

The separation step (b) may be executed in any way known to the skilled person, e.g. by precipitation, filtration, centrifugation, extraction or chromatography and combinations thereof. Preferably the separation step (b) is executed using micro- or ultrafiltration techniques. The pore size of the membranes used in the filtration step, as well as the charge of the membrane may be used to control the separation of the tripeptide MAP and/or the tripeptide ITP. The fractionation of casein protein hydrolysates using charged UF/NF membranes is described in Y. Poilot et al, Journal of Membrane Science 158 (1999) 105-114.

The concentration step (c) may involve nanofiltration or evaporation of the fraction generated by step (b) to yield a highly concentrated liquid. If suitably formulated, e.g. with a low water activity (Aw), a low pH and preferably a preservative such as benzoate or sorbate, such concentrated liquid compositions form an attractive way of storage of the tripeptides according to the invention. Optionally the evaporation step is followed by a drying step e.g. by spray drying or freeze drying to yield a solid containing a high concentration of MAP and/or ITP.

The enzymatic process comprises preferably a single enzyme incubation step. The enzymatic process according to the present invention further relates to the use of a proline specific protease which is preferably free of contaminating enzymatic activities. A proline specific protease is defined as a protease that hydrolyses a peptide bond at the carboxy-terminal side of proline. The preferred proline specific protease is an protease that hydrolyses the peptide bond at the carboxy terminal side of proline and alanine residues. The proline specific protease is preferably capable of hydrolyzing large protein molecules like polypeptides or the protein itself. The process according to the invention has in general an incubation time of less than 24 hours, preferably the incubation time is less than 10 hours and more preferably less than 4 hours. The incubation temperature is in general higher than 30° C., preferably higher than 40° C. and more preferably higher than 50° C.

Another aspect of the present invention is the purification and/or separation of the tripeptides MAP and ITP from a hydrolysed protein. Most of the hydrolysed protein according to the invention is preferably capable to precipitate under selected pH conditions. This purification process comprises altering the pH to the pH whereby most of the hydrolysed and unhydrolysed protein precipitates and separating the precipitated proteins from the (bio-active) tripeptides that remain in solution.

To obtain the present tripeptides with a proline residue at their carboxyterminal end, the use of a protease that can cleave at the carboxyterminal side of proline residues offers a preferred option. Socalled prolyl oligopeptidases (EC 3.4.21.26) have the unique possibility of preferentially cleaving peptides at the carboxyl side of proline residues. Prolyl oligopeptidases also have the possibility to cleave peptides at the carboxyl side of alanine residues, but the latter reaction is less efficient than cleaving peptide bonds involving proline residues. In all adequately characterized proline specific proteases isolated from mammalian as well as microbial sources, a unique peptidase domain has been identified that excludes large peptides from the enzyme's active site. In fact these enzymes are unable to degrade peptides containing more than about 30 amino acid residues so that these enzymes are now referred to as “prolyl oligopeptidases” (Fulop et al: Cell, Vol. 94, 161-170, Jul. 24, 1998). As a consequence these prolyl oligopeptidases require a pre-hydrolysis with other endoproteases before they can exert their hydrolytic action. However, as described in WO 02/45523, even the combination of a prolyl oligopeptidase with such another endoprotease results in hydrolysates characterized by a significantly enhanced proportion of peptides with a carboxyterminal proline residue. Because of this, such hydrolysates form an excellent starting point for the isolation of the tripeptides with in vitro ACE inhibiting effects as well as an improved resistance to gastro-intestinal proteolytic degradation.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. The one-letter code of amino acids used herein is commonly known in the art and can be found in Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989). An endoprotease is defined herein as an enzyme that hydrolyses peptide bonds in a polypeptide in an endo-fasion and belongs to the group EC 3.4. The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25). Exoproteases are defined herein as enzymes that hydrolyze peptide bonds adjacent to a terminal α-amino group (“aminopeptidases”), or a peptide bond between the terminal carboxyl group and the penultimate amino acid (“carboxypeptidases”).

WO 02/45524 describes a proline specific protease obtainable from Aspergillus niger. The A. niger derived enzyme cleaves preferentially at the carboxyterminus of proline, but can also cleave at the carboxyterminus of hydroxyproline and, be it with a lower efficiency, at the carboxyterminus of alanine. WO 02/45524 also teaches that there exists no clear homology between this A. niger derived enzyme and the known prolyl oligopeptidases from other microbial or mammelian sources. In contrast with known prolyl oligopeptidases, the A. niger enzyme has an acid pH optimum. Although the known prolyl oligopeptidases as well as the A. niger derived enzyme are socalled serine proteases, the A. niger enzyme belongs to a completely different subfamily. The secreted A. niger enzyme appears to be a member of family S28 of serine peptidases rather than the S9 family into which most cytosolic prolyl oligopeptidases have been grouped (Rawling, N. D. and Barrett, A. J.; Biochim. Biophys. Acta 1298 (1996) 1-3). The A. niger derived enzyme preparation as used in the process of the present invention is preferably essentially pure meaning that no significant endoproteolytic activity other than the endoproteolytic activity inherent to the pure proline specific endoprotease is present. We also demonstrate that our A. niger derived enzyme preparation preferably used according to the present invention does not contain any exoproteolytic, more specifically aminopeptidolytic side activities. Preferably exoproteolytic activity is absent in the A. niger derived enzyme preparation used in the process of the invention. Experimental proof for the notion that the proline specific endoproteolytic activity is essentially absent in non-recombinant Aspergillus strains can be found in WO 02/45524. Because the process of the present invention is possible by incubating the casein substrate with only the proline specific endoprotease, the optimal incubation conditions like temperature, pH etc. can be easily selected and does not have to be fixed at sub optimal conditions as would be the case if two or more enzymes are applied. Furthermore the formation of unwanted side products as for example additional, non-bio-active peptides or free amino acids leading to brothy off tastes is prevented. Having more degrees of freedom in selecting the reaction conditions makes an easier selection for other criteria possible. For example it is much easier to select now conditions which are less sensitive to microbial infections and to optimise pH conditions relative to subsequent protein precipitation steps. The Aspergillus enzyme is not an oligopeptidase but a true endopeptidase able to hydrolyse intact proteins, large peptides as well as smaller peptide molecules without the need of an accessory endoprotease. This new and surprising finding-opens up the possibility of using the A. niger enzyme for preparing hydrolysates with unprecedented high contents of peptides with a carboxyterminal proline residue because no accessory endoprotease is required. Such new hydrolysates can be prepared from different proteinaceous starting materials be it from vegetable or from animal origin. Examples of such starting materials are caseins, gelatin, fish or egg proteins, wheat gluten, soy and pea protein as well as rice protein and sunflower protein. As sodium is known to play an important role in hypertension, preferred substrates for the production of ACE inhibiting peptides are calcium and potassium rather than sodium salts of these proteins.

The pH optimum of the A. niger derived prolyl endoprotease is around 4.3. Because of this low pH optimum incubating bovine milk caseinate with the A. niger derived prolyl endoprotease is not self-evident. Bovine milk caseinate will precipitate if the pH drops below 6.0 but at pH 6.0 the A. niger enzyme has a limited activity only. Even under this rather unfavorable condition an incubation with the A. niger derived prolyl endoprotease can yield several known ACE inhibiting peptides such as IPP and LPP. Quite surprisingly no VPP is produced under these conditions. Bovine milk casein incorporates a number of different proteins including beta-casein and kappa-casein. According to the known amino sequences beta-casein encompasses the ACE inhibitory tripeptides IPP, VPP and LPP. Kappa-casein encompasses IPP only. The fact that the A. niger derived enzyme does not contain any measurable aminopeptidase activity strongly suggests that the IPP formed is released from the -A107-I108-P109-P110— sequence present in kappa-caseine. Presumably the peptide bond carboxyterminal of IPP is cleaved by the main activity of the A. niger derived prolyl endoprotease whereas cleavage of the preceding Ala-Ile bond is accomplished by its Ala-specific side activity. Similarly the absence of VPP can be explained on the basis of the absence of aminopeptidase side activity. VPP is contained in beta-casein in the sequence -P₈₁V₈₂-V₈₃-V₈₄-P₈₅-P₈₆-. So the proline specific endoprotease excises the VVVPP sequence but is unable to release VPP.

These results are obtained upon incubating the caseinate with the A. niger derived endoprotease in a simple one-step enzyme process. Aqueous solutions containing protein are highly susceptible for microbial infections, especially if kept for many hours at pH values above 5.0 and at temperatures of 50 degrees C. or below. Especially microbial toxins that can be produced during such prolonged incubation steps and are likely to survive subsequent heating steps and form a potential threat to food grade processes. The present invention preferably uses an incubation temperature above 50 degrees C. In combination with the one-step enzyme process in which the enzyme incubation is carried out for a period less than 24 hours, preferably less than 8 hours, more preferably less than 4 hours, the process according to the invention offers the advantage of an improved microbiological stability. Using the present enzyme-substrate ratio in combination with the high temperature conditions, the excision of IPP and LPP is completed within a 3 hours incubation period.

Because the ACE inhibiting peptides IPP and LPP can be excised from casein using a single, essentially pure endoprotease, the present invention results in a smaller number of water soluble peptides than in the prior art processes. Among these water soluble peptides IPP an LPP are present in major amounts. This is especially important in case a high concentration of ACE inhibiting tripeptides is needed without many other, often less active compounds.

According to the present process preferably at least 20%, more preferably at least 30%, most preferably at least 40% of an -I-P-P- or an -L-P-P-sequence present in a protein is converted into the tripeptide IPP or LPP, respectively.

In the Examples we illustrate the 5-fold purification effect of the bio-active peptides by a new and surprising purification step. The basis of this purification process is formed by the unique properties of the A. niger derived proline specific endoprotease. Incubation with this enzyme releases the most bio-active parts of the substrate molecule in the form of water-soluble tripeptides. The non- or less-bioactive parts of the substrate molecule remain to a large extent in non-cleaved and therefore much larger peptide or polypeptide parts of the substrate molecules. Due to the limited water solubilities of these larger peptide or polypeptide parts under selected pH conditions, these non- or less bioactive parts of the substrate molecule are easily separated from the much more soluble bio-active tripeptides. In this process the initial hydrolysate is formed during the brief enzyme incubation period at 55 degrees C., pH 6.0 and is then optionally heated to a temperature above 80 degrees C. to kill all contaminating microorganisms and to inactivate the A. niger derived prolyl endopeptidase.

Subsequently the hydrolysate is acidified to realise a pH drop to 4.5 or at least below 5.0. At this pH value, which cannot be used to inactivate the A. niger derived prolyl endopeptidase because it represents the optimum condition for the enzyme, all large peptides from the caseinate precipitate so that only the smaller peptides remain in solution. As the precipitated caseinates can be easily removed by decantation or a filtration step or a low speed (i.e. below 5000 rpm) centrifugation, the aqueous phase contains a high proportion of bioactive peptides relative to the amount of protein present. According to Kjeldahl data 80 to 70% of the caseinate protein is removed by the low speed centrifugation step which implies a four- to five-fold purification of the ACE inhibiting peptides. We have found that this purification principle can be advantageously applied to obtain biologically active peptides obtained from proteinaceous material other than casein as well. Also not only enzymatically produced hydrolysates but also proteins that are fermentated by suitable microorganisms can be separated and purified according to the present process. Incubating enzyme and substrate at a pH value close to where the substrate will precipitate and where the enzyme is still active, will permit this purification step. Due to the low pH optimum of the A. niger derived prolyl endoprotease, substrate precipitations in the range between pH 1.5 to 6.5 can be considered. In view of their specific precipitation behaviour, gluten precipitations above pH 3.5, sun flower protein precipitations above pH 4.0 and below pH 6.0, egg white precipitations above pH 3.5 and below pH 5.0 form examples of conditions whereby the hydrolysed protein precipitates and the precipitated proteins can be separated from the hydrolysed protein or peptides.

After decantation, filtration or low speed centrifugation, the supernatants containing the biologically active peptides can be recovered in a purified state. A subsequent evaporation and spray drying step will yield an economical route for obtaining a food grade paste or powder with a high bio-activity. Upon the digestion of caseinates according to the process as described, a white and odourless powder with a high concentration of ACE inhibiting peptides, is obtained. Alternatively evaporation or nanofiltration can be used to further concentrate the bio-active peptides. The proper formulation of such a concentrate by increasing the water activity (Aw) in combination with a pH adjustment and the addition of a food grade preservative like a benzoate or a sorbate will yield a microbiologically stabilized, food grade, liquid concentrate of the blood pressure lowering peptides. If appropriately diluted to the right tripeptide concentration, a versatile starting material is obtained suitable for endowing all kinds of foods and beverages with ACE inhibiting properties. If required, the supernatant obtained after the decantation, filtration or low speed centrifugation can be further processed to improve the palatability of the final product. For example, the supernatant can be contacted with powdered activated charcharcoal followed by a filtration step to remove the charcoal. To minimise bitterness of the final product, the supernatant obtained after the decantation, filtration or low speed centrifugation can also be subjected to an incubation with another protease, such as subtilisin, trypsin, a neutral protease or a glutamate-specific endoprotease. If required, the concentration of the bioactive ingredients MAP and/or ITP can be increased even further by subsequent purification steps in which use is made of the specific hydrophilic/hydropholic character of the tripeptides MAP and ITP. Preferred purification methods include nanofiltration (separation on size), extraction for example with hexane or butanol followed by evaporation/precipitation or contacting the acidified hydrolysate as obtained with chromatographic resins from the Amberlite XAD range (Roehm). Also butyl-sepharose resins as supplied by Pharmacia can be used.

In another Example we describe the identification of the new ACE inhibiting peptides MAP and ITP in a casein hydrolysate prepared using the A. niger derived proline specific endoprotease in combination with the new peptide purification process. Only the use of this single and (essentially pure) endoprotease in combination with the removal of a large proportion of the non-bio-active peptides and highly sophisticated separation and identification equipment has allowed us to trace and identify these new ACE inhibiting tripeptides. In the casein derived bioactive peptides (CDBAP) prepared according to the Examples (after precipitation), the tripeptides MAP and ITP were identified in quantities corresponding with 2.9 mg MAP/gram CDBAP (4.8 mg MAP/gram protein in CDBAP) and 0.9 mg ITP/gram CDBAP (1.4 mg ITP/gram protein in CDBAP). A further characteristic for CDBAP is its extraordinary high proline content of 24% on molar basis. The tests described in this Example 7 illustrate the very low IC₅₀ values for the two new tripeptides in the Modified Matsui test i.e. 0.5 micromol/l for MAP and 10 micromol/l for ITP. This finding is even more surprising if we realize that IPP, one of the most effective natural ACE inhibiting peptides known, has an IC₅₀ value in this Modified Matsui test of 2.0 micromol/l.

According to the present process preferably at least 20%, more preferably at least 30%, most preferably at least 40% of an -M-A-P- or an -I-T-P-sequence present in a protein is converted into the tripeptide MAP or ITP, respectively. The usefulness of the newly identified ACE inhibiting peptides MAP and ITP is further illustrated in the Examples. In the latter Example we show that both peptides survive incubation conditions simulating the digestive conditions typically found in the gastro-intestinal tract. On the basis of these data we conclude that the novel tripeptides are likely to survive in the mammalian (for example human) gastrointestinal tract implying a considerable economic potential if used to treat hypertension.

In the Examples we demonstrate that the superior ACE inhibiting peptide MAP cannot only be produced in enzymatic hydrolysis experiments but is also detectable in milk preparations fermented with an appropriate food grade microorganism. However, we have been unable to demonstrate the presence of peptide ITP in such a fermented product.

The peptides MAP and/or ITP as obtained either before or after an additional (for example chromatographic purification steps may be used for the incorporation into food products that are widely consumed on a regular basis. Examples of such products are margarines, spreads, various dairy products such as butter or yoghurts or milk or whey containing beverages. Although such compositions are typically administered to human beings, they may also be administered to animals, preferably mammals, to relief hypertension. Furthermore the high concentration of ACE inhibitors in the products as obtained makes these products very useful for the incorporation into dietary supplements in the form off pills, tablets or highly concentrated solutions or pastes or powders. Slow release dietary supplements that will ensure a continuous release of the ACE inhibiting peptides are of particular interest. The MAP and/or ITP peptides according to the invention may be formulated as a dry powder in, for example, a pill, a tablet, a granule, a sachet or a capsule. Alternatively the enzymes according to the invention may be formulated as a liquid in, for example, a syrup or a capsule. The compositions used in the various formulations and containing the enzymes according to the invention may also incorporate at least one compound of the group consisting of a physiologically acceptable carrier, adjuvant, excipient, stabiliser, buffer and diluant which terms are used in their ordinary sense to indicate substances that assist in the packaging, delivery, absorption, stabilisation, or, in the case of an adjuvant, enhancing the physiological effect of the enzymes. The relevant background on the various compounds that can be used in combination with the enzymes according to the invention in a powdered form can be found in “Pharmaceutical Dosage Forms”, second edition, Volumes 1, 2 and 3, ISBN 0-8247-8044-2 Marcel Dekker, Inc. Although the ACE inhibiting peptides according to the invention formulated as a dry powder can be stored for rather long periods, contact with moisture or humid air should be avoided by choosing suitable packaging such as for example an aluminium blister. A relatively new oral application form is the use of various types of gelatin capsules or gelatin based tablets.

In view of the relevance of natural ACE inhibiting peptides to fight hypertension the present new and cost effective route offers an attractive starting point for mildly hypotensive alimentary or even veterinary products. Because the present route also includes a surprisingly simple purification step, the possibilities for blood pressure lowering concentrated dietary supplements are also enlarged.

By the proline specific endo protease according to the invention or used according to the invention is meant the polypeptide as mentioned in claims 1-5, 11 and 13 of WO 02/45524. Therefore this proline specific endo protease is a polypeptide which has proline specific endoproteolytic activity, selected from the group consisting of:

(a) a polypeptide which has an amino acid sequence which has at least 40% amino acid sequence identity with amino acids 1 to 526 of SEQ ID NO:2 or a fragment thereof;
(b) a polypeptide which is encoded by a polynucleotide which hybridizes under low stringency conditions with (i) the nucleic acid sequence of SEQ ID NO:1 or a fragment thereof which is at least 80% or 90% identical over 60, preferably over 100 nucleotides, more preferably at least 90% identical over 200 nucleotides, or (ii) a nucleic acid sequence complementary to the nucleic acid sequence of SEQ ID NO:1. The SEQ ID NO:1 and SEQ ID NO:2 as shown in WO 02/45524. Preferably the polypeptide is in isolated form.

The preferred polypeptide used according to the present invention has an amino acid sequence which has at least 50%, preferably at least 60%, preferably at least 65%, preferably at least 70%, more preferably at least 80%, even more preferably at least 90%, most preferably at least 95%, and even most preferably at least about 97% identity with amino acids 1 to 526 of SEQ ID NO: 2 or comprising the amino acid sequence of SEQ ID NO:2.

Preferably the polypeptide is encoded by a polynucleotide that hybridizes under low stringency conditions, more preferably medium stringency conditions, and most preferably high stringency conditions, with (i) the nucleic acid sequence of SEQ ID NO:1 or a fragment thereof, or (ii) a nucleic acid sequence complementary to the nucleic acid sequence of SEQ ID NO: 1.

The term “capable of hybridizing” means that the target polynucleotide of the invention can hybridize to the nucleic acid used as a probe (for example, the nucleotide sequence set forth in SEQ. ID NO: 1, or a fragment thereof, or the complement of SEQ ID NO: 1) at a level significantly above background. The invention also includes the polynucleotides that encode the proline specific endoprotease of the invention, as well as nucleotide sequences which are complementary thereto. The nucleotide sequence may be RNA or DNA, including genomic DNA, synthetic DNA or cDNA. Preferably, the nucleotide sequence is DNA and most preferably, a genomic DNA sequence. Typically, a polynucleotide of the invention comprises a contiguous sequence of nucleotides which is capable of hybridizing under selective conditions to the coding sequence or the complement of the coding sequence of SEQ ID NO: 1. Such nucleotides can be synthesized according to methods well known in the art.

A polynucleotide of the invention can hybridize to the coding sequence or the complement of the coding sequence of SEQ ID NO:1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is typically at least 10 fold, preferably at least 20 fold, more preferably at least 50 fold, and even more preferably at least 100 fold, as intense as interactions between other polynucleotides and the coding sequence of SEQ ID NO: 1. The intensity of interaction may be measured, for example, by radiolabelling the probe, for example with 32P. Selective hybridization may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.3M sodium chloride and 0.03M sodium citrate at about 60° C.).

The UWGCG Package provides the BESTFIT program which may be used to calculate identity (for example used on its default settings).

The PILEUP and BLAST N algorithms can also be used to calculate sequence identity or to line up sequences (such as identifying equivalent or corresponding sequences, for example on their default settings).

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold. These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix alignments (B) of 50, expectation (E) of 10, M=5, N=4, and a comparison of both strands.

The BLAST algorithm performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The strains of the genus Aspergillus have a food grade status and enzymes derived from these micro-organisms are known to be from an unsuspect food grade source. According to another preferred embodiment, the enzyme is secreted by its producing cell rather than a non-secreted, socalled cytosolic enzyme. In this way enzymes can be recovered from the cell broth in an essentially pure state without expensive purification steps. Preferably the enzyme has a high affinity towards its substrate under the prevailing pH and temperature conditions.

The nutraceutical products according to the invention may be of any food type. They may comprise common food ingredients in addition to the food product, such as flavour, sugar, fruits, minerals, vitamins, stabilisers, thickeners, etc. in appropriate amounts.

Preferably, the nutraceutical product comprises 50-200 mmol/kg K⁺ and/or 15-60 mmol/kg Ca²⁺ and/or 6-25 mmol/kg Mg²⁺ more preferably, 100-150 mmol/kg K⁺ and/or 30-50 mmol/kg Ca²⁺ and/or 10-25 mmol/kg Mg²⁺ and most preferably 110-135 mmol/kg K⁺ and/or 35-45 mmol/kg Ca²⁺ and/or 13-20 mmol/kg Mg²⁺. These cations have a beneficial effect of further lowering blood pressure when incorporated in the nutraceutical products according to the invention.

Advantageously the nutraceutical product comprises one or more B-vitamins.

The B-vitamin folic acid is known to participate in the metabolism of homocysteine, an amino acid in the human diet. For a number of years, high homocysteine levels have been correlated to high incidence of cardiovascular disease. It is thought that lowering homocysteine may reduce the risk of cardiovascular disease.

Vitamins B6 and B12 are known to interfere with the biosynthesis of purine and thiamine, to participate in the synthesis of the methyl group in the process of homocysteine methylation for producing methionine and in several growth processes. Vitamin B6 (pyridoxine hydrochloride) is a known vitamin supplement. Vitamin B12 (cyanobalamin) contributes to the health of the nervous system and is involved in the production of red blood cells. It is also known as a vitamin in food supplements.

Because of their combined positive effect on cardiovascular disease risk reduction, it is preferred that products according to the invention comprises vitamin B6 and vitamin B12 and folic acid.

The amount of the B-vitamins in the nutraceutical product may be calculated by the skilled person based daily amounts of these B-vitamins given herein: Folic acid: 200-800 Mg/day, preferably 200-400 μg/day; Vitamin B6: 0.2-2 mg/day, preferably 05-1 mg/day and Vitamin B12: 0.5-4 μg/day, preferably 1-2 μg/day.

Preferably, the nutraceutical product comprises from 3 to 25 wt % sterol, more preferred from 7 to 15 wt % sterol. The advantage of the incorporation of sterol is that it will cause reduction of the level of LDL-cholesterol in human blood, which will result in reduction of cardiovascular risk.

Where reference is made to sterol this includes the saturated stanols and esterified derivatives of sterol/stanol or mixtures of any of these.

In this application where reference is made to sterolester, this also includes their saturated derivatives, the stanol esters, and combinations of sterol- and stanol esters.

Sterols or phytosterols, also known as plant sterols or vegetable sterols can be classified in three groups, 4-desmethylsterols, 4-monomethylsterols and 4,4′-dimethylsterols. In oils they mainly exist as free sterols and sterol esters of fatty acids although sterol glucosides and acylated sterol glucosides are also present. There are three major phytosterols namely beta-sitosterol, stigmasterol and campesterol.

Schematic drawings of the components meant are as given in “Influence of Processing on Sterols of Edible Vegetable Oils”, S. P. Kochhar; Prog. Lipid Res. 22: pp. 161-188. The respective 5 alpha-saturated derivatives such as sitostanol, campestanol and ergostanol and their derivatives are in this specification referred to as stanols. Preferably the (optionally esterified) sterol or stanol is selected from the group comprising fatty acid ester of 9-sitosterol, 9-sitostanol, campesterol, campestanol, stigmasterol, brassicasterol, brassicastanol or a mixture thereof.

The sterols or stanols are optionally at least partly esterified with a fatty acid. Preferably the sterols or stanols are esterified with one or more C₂₋₂₂ fatty acids. For the purpose of the invention the term C₂₋₂₂ fatty acid refers to any molecule comprising a C₂₋₂₂ main chain and at least one acid group. Although not preferred within the present context the C₂₋₂₂ main chain may be partially substituted or side chains may be present. Preferably, however the C₂₋₂₂ fatty acids are linear molecules comprising one or two acid group(s) as end group(s). Most preferred are linear C₈₋₂₂ fatty acids as these occur in natural oils.

Suitable examples of any such fatty acids are acetic acid, propionic acid, butyric acid, caproic acid, caprylic acid, capric acid. Other suitable acids are for example citric acid, lactic acid, oxalic acid and maleic acid. Most preferred are myristic acid, lauric acid, palmitic acid, stearic acid, arachidic acid, behenic acid, oleic acid, cetoleic acid, erucic acid, elaidic acid, linoleic acid and linolenic acid. When desired a mixture of fatty acids may be used for esterification of the sterols or stanols. For example, it is possible to use a naturally occurring fat or oil as a source of the fatty acid and to carry out the esterification via an interesterification reaction.

The above described nutraceutical ingredients, contributing to increasing cardiovascular health, K+, Ca2+ and Mg2+, B-vitamins (folic acid, B6, B12) and sterols are herein collectively referred to as heart health ingredients.

The following Examples illustrate the invention further.

A. Pharmaceutical compositions may be prepared by conventional formulation procedures using the ingredients specified below:

### EXAMPLE 1

### Soft Gelatin Capsule

Soft gelatin capsules are prepared by conventional procedures using ingredients specified below:

Active ingredients: MAP and/or ITP 0.1 g, protein hydrolysates 0.3 g

Other ingredients: glycerol, water, gelatin, vegetable oil

### EXAMPLE 2

### Hard Gelatin Capsule

Hard gelatin capsules are prepared by conventional procedures using ingredients specified below:

Active ingredients: MAP and/or ITP 0.3 g, protein hydrolysates 0.7 g

Other ingredients:

Fillers: lactose or cellulose or cellulose derivatives q.s

Lubricant: magnesium stearate if necessary (0.5%)

### EXAMPLE 3

### Tablet

Tablets are prepared by conventional procedures using ingredients specified below:

Active ingredients: MAP and/or ITP 0.4 g, unhydrolysed protein 0.4 g

Other ingredients: microcrystalline cellulose, silicone dioxide (SiO₂), magnesium stearate, crosscarmellose sodium.

B. Food items may be prepared by conventional procedures using ingredients specified below:

### EXAMPLE 4

### Soft Drink with 30% Juice

Typical serving: 240 ml

Active ingredients:

MAP and/or ITP and protein hydrolysates and maltodextrin as a carbohydrate source are incorporated in this food item:

MAP and/or ITP: 0.5-5 g/per serving

Protein hydrolysates: 1.5-15 g/per serving

Maltodextrin: 3-30 g/per serving

I. A Soft Drink Compound is Prepared from the Following Ingredients

Juice concentrates and water soluble flavors
[g]

### 1.1 Orange Concentrate

| | |
|---|---|
| 60.3 °Brix, 5.15% acidity | 657.99 |
| Lemon concentrate | |
| 43.5 °Brix, 32.7% acidity | 95.96 |
| Orange flavor, water soluble | 13.43 |
| Apricot flavor, water soluble | 6.71 |
| Water | 26.46 |

### 1.2 Color

| | |
|---|---|
| β-Carotene 10% CWS | 0.89 |
| Water | 67.65 |

### 1.3 Acid and Antioxidant

| | |
|---|---|
| Ascorbic acid | 4.11 |
| Citric acid anhydrous | 0.69 |
| Water | 43.18 |

### 1.4 Stabilizers

| | |
|---|---|
| Pectin | 0.20 |
| Sodium benzoate | 2.74 |
| Water | 65.60 |

### 1.5 Oil Soluble Flavors

| | |
|---|---|
| Orange flavor, oil soluble | 0.34 |
| Orange oil distilled | 0.34 |

### 1.6 Active Ingredients

Active ingredients (this means the active ingredient mentioned above: MAP and/or ITP and protein hydrolysates and maltodextrin in the concentrations mentioned above.

Fruit juice concentrates and water soluble flavors are mixed without incorporation of air. The color is dissolved in deionized water. Ascorbic acid and citric acid is dissolved in water. Sodium benzoate is dissolved in water. The pectin is added under stirring and dissolved while boiling. The solution is cooled down. Orange oil and oil soluble flavors are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenizing using a Turrax and then a high-pressure homogenizer (p₁=200 bar, p₂=50 bar).

II. A Bottling Syrup is Prepared from the Following Ingredients:

| | [g] |
|---|---|
| Softdrink compound | 74.50 |
| Water | 50.00 |
| Sugar syrup 60° Brix | 150.00 |

The ingredients of the bottling syrup are mixed together. The bottling syrup is diluted with water to 1 l of ready to drink beverage.

### Variations:

Instead of using sodium benzoate, the beverage may be pasteurized. The beverage may also be carbonized.

### EXAMPLE 5

### Incubating Potassium Caseinate with the Proline Specific Endoprotease from A. niger Quickly Yields IPP and LPP but no VPP

In this experiment the overproduced and essentially pure proline specific endoprotease from A. niger was incubated with potassium caseinate to test the liberation of the ACE inhibiting peptides IPP, VPP as well as LPP. The endoprotease used was essentially pure meaning that no significant endoproteolytic activity other than the endoproteolytic activity inherent to the pure proline specific endoprotease (i.e. carboxyterminal cleavage of proline and alanine residues) is present.

To limit sodium intake as the result of the ingestion of ACE inhibiting peptides as much as possible, potassium caseinate was used as the substrate in this incubation.

The caseinate was suspended in water of 65 degrees C. in a concentration of 10% (w/w) protein after which the pH was adjusted to 6.0 using phosphoric acid. Then the suspension was cooled to 55 degrees C. and the A. niger derived proline specific endoprotease was added in a concentration of 4 units/gram of protein (see Materials & Methods section for unit definition). Under continuous stirring this mixture was incubated for 24 hours. No further pH adjustments were carried out during this period. Samples were taken after 1, 2, 3, 4, 8 and 24 hours of incubation. Of each sample enzyme activity was terminated by immediate heating of the sample to 90 degrees C. for 5 minutes. After cooling down the pH of each sample was quickly lowered to 4.5 using phosphoric acid after which the suspension was centrifuged for 5 minutes at 3000 rpm in a Hereaus table top centrifuge. The completely-clear supernatant was used for LC/MS/MS analysis to quantify the peptides VPP, IPP, LPP, WVPP and VVVPPF in the supernatant (see Materials & Methods section).

Bovine milk casein incorporates a number of different proteins including beta-casein and kappa-casein. According to the known amino sequences beta-casein encompasses the ACE inhibitory tripeptides IPP, VPP and LPP. In beta-casein IPP is contained in the sequence -P₇₁-Q₇₂-N₇₃-I₇₄-P₇₅-P₇₆-, VPP is contained in the sequence -P₈₁-V₈₂-V₈₃-V₈₄-P₈₅-P₈₆- and LPP is contained in the sequence -P₁₅₀-L₁₅₁-P₁₅₂-P₁₅₃-. Kappa-casein, which is present in acid precipitated caseinate preparations in a molar concentration of almost 50% of the beta-casein concentration, encompasses IPP only. In kappa-casein IPP is contained in the sequence -A₁₀₇-I₁₀₈-P₁₀₉-P₁₁₀-. The other protein constituents of casein do not contain either IPP, VPP or LPP.

Tables 2 and 3 show the concentrations of the peptides present in the acidified and centrifuged supernatants as calculated per gram of potassium caseinate added to the incubation mixture. As shown in Table 2, IPP reaches its maximal concentration after 1 hour of incubation. Beyond that the IPP concentration does not increase any further. The formation of the pentapeptide VVVPP shows the same kinetics as the generation of IPP. As theoretically expected, the molar yield of VVVPP is similar to the molar yield of the LPP peptide. The yield of both LPP and VVVPP reach almost 60% of what would be theoretically feasible. The fact that the maximum concentration of LPP is reached only after 3 hours of incubation suggests that cleavage of that particular part of the beta-caseine molecule is perhaps somewhat more difficult. In contrast with VVVPP, the hexapeptide VVVPPF is not formed at all. This observation suggests that the proline specific endoprotease efficiently cleaves the —P—F— bond hereby generating VVVPP. The tripeptide IPP is formed immediately but its molar yield is not more than about a third of the maximal molar yield of either VVVPP or LPP. As the IPP tripeptide is contained in both beta-caseine as in kappa-caseine, this outcome is unexpected. A likely explanation for this observation is that the proline specific protease can generate IPP but from the kappa-caseine moiety of the caseinates only. In view of the relevant amino acid sequence of kappa-caseine this suggests that the -A₁₀₇-I₁₀₈-peptide bond is cleaved by the alanine-specific activity of the enzyme. If true, the amount of IPP liberated reaches approximately 40% of the quantity that is present in kappa-casein, but not more than about 10% of the IPP that is theoretically present in beta plus kappa casein. This cleavage mechanism for the release of IPP also explains why VPP cannot be formed from its precursor molecule WVPP: the required endoproteolytic activity is simply not present within the A. niger derived enzyme preparation used.

TABLE 2
Molar peptide contents of acidified supernatants calculated per
gram of protein added.

| micromole/gram | | | | | | |
|---|---|---|---|---|---|---|
| protein | | IPP | LPP | VPP | VVVPP | VVVPPF |
| K-cas | 1 hr | 2.8 | 4.2 | <0.2 | 8.4 | <0.2 |
| K-cas | 2 hrs | 2.6 | 6.1 | <0.2 | 9.1 | <0.2 |
| K-cas | 3 hrs | 2.6 | 8.4 | <0.2 | 9.1 | <0.2 |
| K-cas | 4 hrs | 2.3 | 8.0 | <0.2 | 8.3 | <0.2 |
| K-cas | 8 hrs | 2.1 | 9.4 | <0.2 | 7.2 | <0.2 |
| K-cas | 24 hrs | 2.0 | 9.5 | 0.4 | 5.5 | <0.2 |

TABLE 3
Peptide concentrations in acidified supernatants calculated in
mg/g protein added.

| milligram/gram | | | | | | |
|---|---|---|---|---|---|---|
| protein | | IPP | LPP | VPP | VVVPP | VVVPPF |
| K-cas | 1 hr | 0.9 | 1.4 | <0.05 | 4.3 | <0.05 |
| K-cas | 2 hrs | 0.8 | 2.0 | <0.05 | 4.6 | <0.05 |
| K-cas | 3 hrs | 0.8 | 2.7 | <0.05 | 4.6 | <0.05 |
| K-cas | 4 hrs | 0.8 | 2.6 | <0.05 | 4.2 | <0.05 |
| K-cas | 8 hrs | 0.7 | 3.0 | <0.05 | 3.6 | <0.05 |
| K-cas | 24 hrs | 0.7 | 3.1 | 0.1 | 2.8 | <0.05 |

### EXAMPLE 6

### Incorporation of an Acid Casein Precipitation Step Results in a 5-Fold Concentration of ACE Inhibiting Peptides

As described in Example 5, potassium caseinate in a concentration of 10% (w/w) protein was subjected to an incubation with the A. niger derived proline specific endoprotease at pH 6.0. After various incubation periods samples were heated to stop further enzyme activity after which the pH was lowered to 4.5 to minimise casein solubility. Non soluble casein molecules were removed by a low speed centrifugation. In Tables 2 and 3 we have provided concentrations of ACE inhibiting peptides calculated on the basis of the starting concentration of 10% protein. However, as the result of the acidification and the subsequent centrifugation step, a large proportion of the protein added has been removed. To take these reduced protein contents of the acidified supernatants into account, nitrogen (Kjeldahl) analyses were carried out. According to the latter data the various supernatants were found to contain the protein levels shown in Table 4.

TABLE 4
Protein contents of acidified supernatants

| | Protein content |
|---|---|
| Sample | (grams/liter) |
| K-cas 1 hr | 21 |
| K-cas 2 hrs | 27 |
| K-cas 3 hrs | 30 |
| K-cas 4 hrs | 34 |
| K-cas 8 hrs | 40 |
| K-cas 24 hrs | 48 |

Taking these data into account, we have recalculated the concentration of the ACE inhibiting peptides present in each supernatant but this time using their actual protein contents. These recalculated data are shown in Table 5.

TABLE 5
Peptide concentrations in acidified supernatants calculated per
gram of protein present.

| Milligram/gram | | | | | | |
|---|---|---|---|---|---|---|
| protein | | VPP | IPP | LPP | VVVPP | VVVPPF |
| K-cas | 1 hr | 0.1 | 4.8 | 7.1 | 22.5 | <0.05 |
| K-cas | 2 hr | 0.1 | 3.4 | 8.0 | 18.9 | <0.05 |
| K-cas | 3 hr | 0.1 | 3.1 | 10.0 | 17.0 | <0.05 |
| K-cas | 4 hr | 0.1 | 2.4 | 8.5 | 13.7 | <0.05 |
| K-cas | 8 hr | 0.1 | 1.9 | 8.4 | 10.0 | <0.05 |
| K-cas | 24 hr | 0.3 | 1.5 | 7.1 | 6.4 | <0.05 |

Comparison of the data presented in Tables 3 and 5 clearly shows that the simple acidification step followed by an industrially feasible decantation, filtration or low speed centrifugation step results in a 5-fold increase in the concentration of the specific ACE inhibiting peptides.

### EXAMPLE 7

### Identification of the Novel and Potent ACE Inhibiting Tripeptides MAP and ITP in Concentrated Casein Hydrolysates

To facilitate a more thorough analysis of bio-active peptides present, the casein hydrolysate obtained by the digestion with pure A. niger derived proline specific endoprotease and purified by acid precipitation was prepared on a preparative scale. To that end 3000 grams of potassium caseinate was suspended in 25 liters of water of 75 degrees C. After a thorough homogenisation the pH was slowly adjusted to 6.0 using diluted phosphoric acid. After cooling down to 55 degrees C., the A. niger derived proline specific endoproteases was added in a concentration of 4 enzyme units/gram caseinate (see Materials & Methods section for unit definition). After an incubation (with stirring) for 3 hours at 55 degrees C., the pH was lowered to 4.5 by slowly adding concentrated phosphoric acid. In this larger scale preparation the heat treatment step to inactivate the proline specific endoprotease at this part of the process was omitted. Then the suspension was quickly cooled to 4 degrees C. and kept overnight (without stirring) at this temperature. The next morning the clear upper layer was decanted and evaporated to reach a level of 40% dry matter. The latter concentrated liquid was subjected to a UHT treatment of 4 seconds at 140 degrees C. and then ultrafiltered at 50 degrees C. After germ filtration, the liquid was spray dried. This material is hereinafter referred to as Casein Derived Bio-Active Peptides (CDBAP). Using the LC/MS procedures outlined in the Materials & Methods section, the IPP, LPP and VPP content of the powdered product was determined. According to its nitrogen content, the powdered product has a protein content of about 60% (using a conversion factor of 6.38). The IPP, LPP and VPP contents of the powder are provided in Table 6. The amino acid composition of the CDBAP product is provided in Table 7. Quite remarkable is the increase of the molar proline content of the spray dried material obtained after acid precipitation: from an initial 12% to approx 24%.

TABLE 6
IPP, LPP and VPP content of CDBAP.

| | | |
|---|---|---|
| IPP | LPP | VPP |

Tripeptide content in mg/gram powder

| | | |
|---|---|---|
| 2.5 | 6.5 | <0.1 |

Tripeptide content in mg/gram protein

| | | |
|---|---|---|
| 4.2 | 10.8 | <0.17 |

TABLE 7
Amino acid composition of the potassium caseinate starting
material and CDBAP (amino acid contents after acid hydrolysis
and shown as percentages of the molar amino acid content).

| | Starting | |
|---|---|---|
| Amino acid | material | CDBAP |
| Asp | 6.5 | 3.2 |
| Glu | 18.9 | 12.5 |
| Asn | — | — |
| Ser | 6.7 | 4.3 |
| Gln | — | — |
| Gly | 3.5 | 3.2 |
| His | 2.2 | 3.7 |
| Arg | 2.8 | 2.3 |
| Thr | 4.3 | 3.0 |
| Ala | 4.5 | 3.4 |
| Pro | 12.3 | 24.1 |
| Tyr | 3.9 | 2.4 |
| Val | 7.1 | 9.6 |
| Met | 2.3 | 3.9 |
| Ile | 5.0 | 4.1 |
| Leu | 9.2 | 9.0 |
| Phe | 4.0 | 3.9 |
| Lys | 6.9 | 7.4 |
| Total | 100 | 100 |

The presence of novel ACE inhibiting peptides in CDBAP was investigated by using 2-dimensional-chromatographic-separation combined with an at-line ACE inhibition assay and mass spectrometry for identification. In the first analysis the peptide mixture was separated on an ODS3 liquid chromatography (LC) column and ACE inhibition profiles were generated from the various fractions obtained. In a second analysis the fractions from the first column showing a high ACE inhibition were further separated on a Biosuite LC column using a different gradient profile. The fractions collected from this second column were split into two parts: one part was used for the at-line ACE inhibition measurement while the other part was subjected to MS and MS-MS analysis to identify the peptides present.

All analyses were performed using an Alliance 2795 HPLC system (Waters, Etten-Leur, the Netherlands) equipped with a dual trace UV-detector. For identification of the peptides the HPLC-system was coupled to a Q-TOF mass spectrometer from the same supplier. In the tests 20 μl of a 10% (w/v) solution of CDBAP in Milli-Q water was injected on a 150×2.1 Inertsil 5 ODS3 column with a particle size of 5 μm (Varian, Middelburg, the Netherlands). Mobile phase A consisted of a 0.1% trifluoroacetic acid (TFA) solution in Milli-Q water. Mobile phase B consisted of a 0.1% TFA solution in acetonitrile. The initial eluent composition was 100% A. The eluent was kept at 100% A for 5 minutes. Then a linear gradient was started in 10 minutes to 5% B, followed by a linear gradient in 10 minutes to 30% B. The column was flushed by raising the concentration of B to 70% in 5 minutes, and was kept at 70% B for another 5 minutes. After this the eluent was changed to 100% A in 1 minute and equilibrated for 9 minutes. The total run time was 50 minutes. The effluent flow was 0.2 ml min⁻¹ and the column temperature was set at 60° C. A UV chromatogram was recorded at 215 nm. Eluent fractions were collected in a 96 well plate using a 1 minute interval time resulting in fraction volumes of 200 μl. The effluent in the wells was neutralised by addition of 80 μl of a 0.05% solution of aqueous ammonium hydroxide (25%). The solvent was evaporated until dryness under nitrogen at 50° C. After this the residue was reconstituted in 40 μl of Milli-Q water and mixed for 1 minute.

For the at-line ACE inhibition assay 27 μl of a 33.4 mU ml⁻¹ ACE (enzyme obtained from Sigma) in phosphate buffered saline (PBS) pH 7.4 with a chloride concentration of 260 mM was added and the mixture was allowed to incubate for 5 minutes on a 96 well plate mixer at 700 RPM. After the incubation period 13 μl of a 0.35 mM hippuric acid-histidine-leucine (HHL) solution in PBS buffer was added and mixed for 1 minute at 700 RPM. The mixture was allowed to react for 60 minutes at 50° C. in a GC-oven. After the reaction the plate was cooled in melting ice.

The 96 well plate was then analysed on a flash-HPLC-column. Of the reaction mixture of each well 30 μl was injected on a Chromlith Flash RP18e 25×4.6 mm HPLC column (Merck, Darmstadt, Germany) equipped with a 10×4.6 mm RP18e guard column from the same supplier. The isocratic mobile phase consisted of a 0.1% solution of TFA in water/acetonitrile 79/21. The eluent flow was 2 ml min⁻¹ and the column temperature was 25° C. The injections were performed with an interval time of 1 minute. Hippuric acid (H) and HHL were monitored at 280 nm. The peak heights of H and HHL were measured and the ACE inhibition (ACEI) of each fraction was calculated according to the equation:

$$ACEI α = \frac{( {DC}_{w} - {DC}_{α} )}{{DC}_{w} * 100}$$

$$ACEI αPercentage inhibition of the analyte$$

$$\begin{matrix}{DC}_{w} & Degree of Cleavage by ACE of HHL to H and HL in water \\ {DC}_{a} & Degree of Cleavage of HHL to H and HL for the analyte\end{matrix}$$

The Degree of Cleavage was calculated by expressing the peak height of H as a fraction of the sum of the peak heights of H and HHL.

The highest ACE inhibition was measured in the fractions eluting between 18 and 26 minutes. This region was collected and re-injected on a 150×2.1 mm Biosuite column with a particle size of 3 μm (Waters, Etten-Leur, the Netherlands). Mobile phase A here consisted of a 0.1% formic acid (FA) solution in Milli-Q water. Mobile phase B consisted of a 0.1% FA solution in methanol. The initial eluent composition was 100% A. The eluent was kept at 100% A for 5 minutes. After this a linear gradient was started in 15 minutes to 5% B, followed by a linear gradient in 30 minutes to 60% B. The eluent was kept at 60% B for another 5 minutes. Finally the eluent was reduced to 100% of mobile phase A in 1 minute and equilibrated for 10 minutes. The total run time was 65 minutes. The eluent flow was 0.2 ml min⁻¹ and the column temperature was set at 60° C. The UV trace was recorded at 215 nm. Fractions were collected from the Biosuite column at 10 seconds interval time. The fractions were again split into two parts, one part was used to measure the activity using the at-line ACE inhibition method described earlier, while the other part was used to identify the active peptides using MS and MS-MS.

Two chromatographic peaks with molecular ions of 326.2080 Da and two other peaks with molecular ions of 330.2029 Da and 318.1488 Da corresponded with the increased ACE inhibition measured in the area between 18 and 26 minutes. Using MS-MS these peptides were identified as the structural isomers IPP and LPP (−0.6 ppm), ITP (−4.8 ppm) and MAP (+2.8 ppm) respectively. The protein sources of the peptides are kappa-casein f108-110 (IPP), β-casein f151-153 (LPP), α-s2-casein f119-121 (ITP) and β-casein f102-104 (MAP). IPP and LPP were reported earlier as ACE inhibiting peptides with IC50 values of 5 and 9.6 μM respectively (Y. Nakamura, M. Yamamoto., K. Sakai., A. Okubo., S. Yamazaki, T. Takano, J. Dairy Sci. 78 (1995) 777-783; Y. Aryoshi, Trends in Food Science and Technol. 4 (1993) 139-144). However, the tripeptides ITP and MAP were, to our knowledge, never before reported as potent ACE inhibiting peptides.

MAP, ITP and IPP were chemically synthesised and the activity of each peptide was measured using a modified Matsui assay described hereafter

Quantification of MAP and ITP in the various samples was performed on a Micromass Quattro II MS instrument operated in the positive electrospray, multiple reaction monitoring mode. The HPLC method used was similar to the one described above. The MS settings (ESI+) were as follows: cone voltage 37 V, capillary voltage 4 kV, drying gas nitrogen at 300 l/h. Source and nebulizer temperature: 100° C. and 250° C., respectively. The synthesized peptides were used to prepare a calibration line using the precursor ion 318.1 and the summed product ions 227.2 and 347.2 for MAP and using the precursor ion 320.2 and the summed product ions 282.2 and 501.2 for ITP. According to these analyses the novel ACE inhibiting tripeptides MAP and ITP are present in the CDBAP product in quantities corresponding with 2.9 mg MAP/gram CDBAP or 4.8 mg MAP/gram protein in CDBAP and 0.9 mg ITP/gram CDBAP en 1.4 mg ITP/gram protein in CDBAP. To determine the ACE inhibition activity of MAP and ITP, the chemically synthesised tripeptides were assayed according to the method of Matsui et al. (Matsui, T. et al. (1992) Biosci. Biotech. Biochem. 56: 517-518) with some minor modifications. The various incubations are shown in Table 8.

TABLE 8
Procedure for Matsui ACE inhibition assay. The components were
added in a 1.5-ml tube with a final volume of 120 μl.

| | Control 1 | Control 2 | Sample 1 | Sample 2 |
|---|---|---|---|---|
| Component | (μl) | (μl) | (μl) | (μl) |
| Hip-His-Leu (3 mM) | 75 | 75 | 75 | 75 |
| H₂O | 25 | 45 | — | 20 |
| Inhibiting | — | — | 25 | 25 |
| peptide | | | | |
| ACE (0.1 U/ml) | 20 | — | 20 | — |

Each one of the four samples contained 75 μl 3 mM hippuryl histidine leucine (Hip-His-Leu, Sigma) dissolved in a 250 mM borate solution containing 200 mM NaCl, pH 8.3. ACE was obtained from Sigma. The mixtures were incubated at 37° C. and stopped after 30 min by adding 125 μl 0.5 M HCl. Subsequently, 225 μl bicine/NaOH solution (1 M NaOH:0.25 M bicine (4:6)) was added, followed by 25 μl 0.1 M TNBS (2,4,6-Trinitrobenzenesulfonic acid, Fluka, Switzerland; in 0.1 M Na₂HPO₄). After incubation for 20 min. at 37° C., 4 ml 4 mM Na₂SO₃ in 0.2 M NaH₂PO₄ was added and the light absorbance at 416 nm was measured with UV/Vis spectrophotometer (Shimadzu UV-1601 with a CPS controller, Netherlands).

The amount of ACE inhibition (ACEI) activity was calculated as a percentage of inhibition compared with the conversion rate of ACE in the absence of an inhibitor according to the following formula:

ACEI (%)=((Control1−Control 2)−(Sample 1−Sample 2))/(Control 1−Control 2))*100 wherein
Control 1=Absorbance without ACE inhibitory component (=max. ACE activity) [AU].
Control 2=Absorbance without ACE inhibitory component and without ACE (background) [AU].
Sample1=Absorbance in the presence of ACE and the ACE inhibitory component [AU].
Sample 2=Absorbance in the presence of the ACE inhibitory component, but without ACE [AU].

The IC₅₀ of the chemically synthesized MAP and ITP tripeptides as obtained are shown in Table 9 together with IC₅₀ values obtained in the at-line measurements used in the screening phase of the experiment. The measurement of chemically synthesized IPP was included as an internal reference for the various measurements.

TABLE 9
ACE inhibition (IC50 values) of MAP, ITP and IPP values
determined by the at-line ACE assay and the modified Matsui
assay.

IC50 value in μM

| | at-line ACE | Modified Matsui |
|---|---|---|
| Peptide | assay | assay |
| MAP | 3.8 | 0.4 |
| ITP | 50 | 10 |
| IPP (reference) | 7.1 | 2 |

### EXAMPLE 8

### Novel ACE Inhibiting Peptides MAP and ITP are Likely to Survive in the Human Gastrointestinal Tract

After consumption, dietary proteins and peptides are exposed to various digestive enzymatic processes in the gastrointestinal tract. In order to assess the stability of the newly identified bioactive peptides in the human gastrointestinal tract, the CDBAP preparation (prepared as described in Example 7) was subjected to a gastro-intestinal treatment (GIT) simulating the digestive conditions typically found in the human body. Samples obtained after various incubation times in the GIT model system were analysed using the on-line HPLC-Bioassay-MS or HRS-MS system to quantify any residual MAP and ITP peptides. The GIT procedure was performed in a standardized mixing device incorporating a 100 ml flask (as supplied by Vankel, US). The temperature of the water bath was set to 37.5° C. and the paddle speed was chosen such that the sample was kept in suspension (100 rpm).

About 3.4 grams of CDBAP (protein level of approx 60%) was dissolved/suspended in 100 ml Milli-Q water. During gastric simulation 5 M HCl was used to decrease the pH. At the end of gastric simulation and during the duodenal phase 5 M NaOH was used to raise the pH.

The CDBAP suspension was preheated to 37.5° C. and 5 ml of the suspension was removed to dissolve 0.31 g of pepsin (Fluka order no. 77161). At t=0 min the 5 ml with the now dissolved pepsin was added back to the suspension. Then the pH of the CDBAP suspension was adjusted slowly by hand using a separate pH meter according to the following scheme:

| | |
|---|---|
| t = 20 min | pH decreased to 3.5 |
| t = 40 min | pH to 3.0 |
| t = 50 min | pH to 2.3 |
| t = 60 min | pH to 1.8 |
| t = 65 min | pH raised to 2.7 |
| t = 75 min | pH to 3.7 |
| t = 80 min | pH to 5.3 |

At t=90 min 0.139 g of 8 times USP pancreatin (Sigma order no. P7545) was carefully mixed in another 5 ml of the CDBAP suspension and immediately added back. The incubation continued according to the following scheme:

| | |
|---|---|
| t = 93 min | pH to 5.5 |
| t = 95 min | pH to 6.3 |
| t = 100 min | pH to 7.1 |

The experiment was stopped at t=125 min and the pH was checked (was still pH 7).

Then the samples were transferred into a beaker and were placed in a microwave till boiling. Subsequently, the samples were transferred into glass tubes and incubated at 95° C. for 60 min to inactivate all protease activity. After cooling the samples were put in Falcon tubes and centrifuged for 10 min at 3000×g. The supernatant was freeze dried. The total N concentration of the powder as obtained was determined and converted to protein level using the Kjeldahl factor of casein (6.38). According to these data the protein level of the CDBAP preparation after the GIT procedure was 48.4%. The levels of MAP and ITP surviving the proteolytic treatment according to the GIT procedure were determined as described in Example 7 and the data obtained are shown in Table 10.

According to the results of the experiment both MAP and ITP exhibit a high resistance against GIT digestion. In combination with the low IC₅₀ values for these tripeptides (also as determined in Example 7), the data suggest considerable potential for the two novel ACE inhibiting peptides as blood pressure lowering peptides.

TABLE 10
Concentrations of MAP and ITP before and after passage through
a simulated human gastro-intestinal tract (GIT procedure)

Concentration
in μg g⁻¹ powder

| Sample | MAP | ITP |
|---|---|---|
| CDBAP (Example | 2851.4 | 903.7 |
| 7) | | |
| CDBAP after | 3095.8 | 889.1 |
| GIT | | |

### EXAMPLE 9

### Simulated In-Vitro Gastro-Intestinal Digestion of Synthetic Map and ITP

In order to measure stability of the peptides in the gastrointestinal tract (GI) micro-dissolution was used. This following test was used to test the GI stability of MAP and ITP.

### Components:

For the dissolution the following solutions were used:

0.1 mol/l HCl
1 mol/l NaHCO₃

Simulated gastric fluid;

1.0 g sodium chloride en 3.5 ml 0.1 mol/l HCl in 500 ml water (degassed in sonification bath, 10 min.)

Enzymes gastric conditions (amounts needed in 1 ml total volume):

2.9 mg Pepsine en 0.45 mg Amano Lipase-FAP15 in 50 μl simulated gastric fluid

Enzymes intestinal conditions (amounts needed in 1 ml total volume):

9 mg Pancreatine (Sigma P8096) en 0.125 mg bile extract in 50 μl 1.0 mol/l NaHCO₃

### Procedure:

Gastric conditions:
Each vial was filled with:
0.82 ml simulated gastric fluid+70 μl MilliQ+10 μg (10×diluted) Mixture 1,
take a sample when T=37.5° C. (t=0), add 50 μl pepsine/lipase mixture (shake).
The pH is measured and adjusted to 3.5 with 0.1 mol/l HCl
Incubation for 60 minutes, after 60′ a sample is taken.
   Intestinal conditions:
50 μl pancreatine mixture is added, the pH is measured and adjusted to 6.8 with
HCl.
Samples are taken at 5′, 30′ en 60′ after the addition of pancreatine (shake).
All samples are kept at 95° C. for 60 minutes to stop the enzyme from being active.
After cooling the samples were stored at −20° C. until analysis.
The samples were centrifuged and analyzed with HPLC-MRM-MS.

For tables 11 and 12 the measured concentration of the peptides is given in ng/ml, calculated to the relative concentration of MAP.

TABLE 11
Simulated in-vitro gastro-intestinal digestion of synthetic MAP -
1 microgram/ml

| | | | % | % | |
|---|---|---|---|---|---|
| Time | a | b | remaining | remaining | % average |
| (minutes) | conc | ng/ml | trial 1 | trial 2 | remaining |
| 0 | — | 2962.5 | 100 | 100 | 100 |
| 30 | — | 2760 | — | 93 | 93 |
| 60 | 1902.6 | — | 64 | — | 62 |
| 65 | 1384.6 | 1654.1 | 47 | 56 | 51 |
| 75 | 2282.2 | 1608.3 | 43 | 54 | 49 |
| 90 | 730.5 | 911.6 | 25 | 31 | 28 |
| 120 | 377.2 | 503.3 | 13 | 17 | 15 |

Where — is indicated this denotes that measurements were not taken.

TABLE 12
Simulated in-vitro gastro-intestinal digestion of synthetic MAP -
10 microgram/ml

| | | | % | % | |
|---|---|---|---|---|---|
| Time | a | b | remaining | remaining | % average |
| (minutes) | conc | ng/ml | trial 1 | trial 2 | remaining |
| 0 | — | 82499.2 | 100 | 100 | 100 |
| 30 | 50635.6 | 76600.6 | 61 | 93 | 77 |
| 65 | 28492.5 | 33339.1 | 35 | 40 | 37 |
| 75 | 21936.4 | 21991.9 | 27 | 27 | 27 |
| 90 | 7588.3 | 10490.8 | 9 | 13 | 11 |
| 120 | 2810.6 | 2661.8 | 3 | 3 | 3 |

Where — is indicated this denotes that measurements were not taken.

TABLE 13
Simulated in-vitro gastro-intestinal digestion of synthetic ITP -
1 microgram/ml

| | | | % | % | |
|---|---|---|---|---|---|
| Time | a | b | remaining | remaining | % average |
| (minutes) | conc | ng/ml | trial 1 | trial 2 | remaining |
| 0 | 1325.201 | 901.297 | 100 | 100 | 100 |
| 30 | 1236.423 | 952.165 | 93 | 106 | 99 |
| 60 | 950.665 | 893.015 | 72 | 99 | 85 |
| 65 | 722.452 | 677.991 | 55 | 75 | 65 |
| 75 | 707.693 | 698.078 | 43 | 77 | 65 |
| 90 | 603.143 | 704.863 | 46 | 78 | 62 |
| 120 | 701.749 | 678.751 | 53 | 75 | 64 |

Where — is indicated this denotes that measurements were not taken.

TABLE 14
Simulated in-vitro gastro-intestinal digestion of synthetic ITP -
10 microgram/ml

| | | | % | % | |
|---|---|---|---|---|---|
| Time | a | b | remaining | remaining | % average |
| (minutes) | conc | ng/ml | trial 1 | trial 2 | remaining |
| 0 | 11230.3 | 9388.467 | 100 | 100 | 100 |
| 30 | 8725.687 | 7884.828 | 78 | 84 | 81 |
| 60 | 8542.271 | 9951.495 | 76 | 106 | 91 |
| 65 | 6739.74 | 8504.414 | 60 | 91 | 75 |
| 75 | 7016.45 | 6052.258 | 62 | 64 | 63 |
| 90 | 7212.26 | 5660.004 | 64 | 60 | 62 |
| 120 | 5168.85 | — | 46 | — | 46 |

Where — is indicated this denotes that measurements were not taken.

The above results demonstrate that the tripeptide MAP exhibits reasonably good stability under gastro-intestinal conditions especially after 1 hour under stomach conditions. Although, MAP undergoes further degradation before reaching the end of the gut, most peptides are absorbed shortly after the stomach i.e. in the duodenum and the proximal part of the jejunum. However, it is believed that MAP is protected against this degradation in the presence of other peptides within the casein hydrolysate.

The results also demonstrate the excellent stability under gastro-intestinal conditions of ITP. This excellent stability may compensate for the somewhat lower potency of ITP as an ACE inhibitor.

These results demonstrate that the tripeptide MAP exhibits reasonably good stability under gastro-intestinal conditions especially after 1 hour under stomach conditions. However, it does undergo further degradation before reaching the end of the gut. However, it is believed that MAP is protected against this degradation in the presence of other peptides within the casein hydrolysate; this explains the apparent differences in stability for MAP shown in examples 8 and 9.

### EXAMPLE 10

### Preparation of a Map Containing Fermented Milk

As described in Example 7 the highly potent ACE inhibiting tripeptide MAP was identified in a casein hydrolysate prepared according to the enzymatic procedure described in Example 7. However, we wondered whether the MAP tripeptide could also be obtained using the more common approach of fermenting skim milk. To test this use was made of a lactobacillus strain characterized by an API50CHL strip (available from bioMerieux SA, 69280 Marcy-l'Etoile, France). The strain used was able to ferment D-glucose, D-fructose, D-mannose, N-acetyl glucosamine, maltose, lactose, sucrose and trehalose. According to the APILAB Plus databank (version 5.0; also available from bioMerieux) the strain was characterized as a Lactobacillus delbrueckii subsp. Lactis 05-14. The strain was deposited at the Centraal Bureau voor Schimmelculturen, Baarn, The Netherlands (CBS109270).

To prepare a preculture for the actual fermentation experiment, sterile skim milk (Yopper ex Campina, Netherlands) was inoculated with 2 to 4% of a culture of the Lactobacillus delbrueckii strain and grown for 24 hours at 37 degrees C.

In the actual fermentation experiment, reconstituted milk of 4.2% MPC-80 (Campina, Netherlands), 0.5% lactose and 0.3% Lacprodan 80 (Campina, Netherlands), was pasteurised for 2 min at 80 degrees. After cooling down the milk was inoculated with 2 wt % of the preculture and fermentation was performed in 150 ml jars under static conditions and performed without pH control at 40° C.

After 24 hours a sample was taken and centrifuged for 10 min at 14.000 g. The pH of the sample obtained was 5.3 and the MAP concentration 18.3 mg/L. However, ITP could not be detected in the fermented milk.

1. (canceled)

2. (canceled)

3. (canceled)

4. (canceled)

5. (canceled)

6. (canceled)

7. A method of treatment of type 1 and 2 diabetes, and for the prevention of type 2 diabetes in those individuals with pre-diabetes, or impaired glucose tolerance (IGT) which comprises administering to a subject in need of such treatment MAP and/or ITP or a salt of MAP and/or a salt of ITP.

8. A method of treatment of people that suffer of hypertension or heart failure or the prevention thereof which comprises administering to a subject in need of such treatment MAP and/or ITP or a salt of MAP and/or a salt of ITP.

9. (canceled)

10. (canceled)

11. (canceled)

12. (canceled)

13. The process according to claim 8 wherein MAP and/or ITP is in the form of a dietary supplement.

14. (canceled)

15. (canceled)

16. (canceled)

17. (canceled)

18. (canceled)

19. Food comprising MAP and or ITP or a salt of MAP and/or a salt of ITP as active ingredient.

20. (canceled)

21. (canceled)

22. A composition comprising MAP and/or ITP or a salt of MAP and/or a salt of ITP as a topical agent preferably for use in personal care.

23. A composition according to claim 22 which is a lotion, a gel or an emulsion.

24. (canceled)

25. (canceled)

26. A process of producing MAP and/or ITP which comprises the fermentation of a suitable protein by a suitable micro organism which is capable of producing MAP and/or ITP from the protein.

27. (canceled)
